(19) United States
(12) Patent Application Publication
(10) Pub. No.: US 2017/0128258 A1
(43) Pub. Date: May 11, 2017

(54) CRYOTHERAPY DEVICES AND METHODS WITH ALTERNATING COOLING AND HEATING TO LIMIT ISCHEMIC INJURY AND TO ENHANCE WOUND HEALING

(71) Applicants: Kenneth R. Diller, Elgin, TX (US); Sepideh Khoshnevis, Austin, TX (US); Kenneth H. Lee, Arlington, TX (US); Wesley R. Hejl, Pflugerville, TX (US)

(72) Inventors: Kenneth R. Diller, Elgin, TX (US); Sepideh Khoshnevis, Austin, TX (US); Kenneth H. Lee, Arlington, TX (US); Wesley R. Hejl, Pflugerville, TX (US)

(21) Appl. No.: 15/322,912

(22) PCT Filed: Jul. 2, 2015
(86) PCT No.: PCT/US15/38971

§ 371 Date: Dec. 29, 2016

Related U.S. Application Data
(60) Provisional application No. 62/020,100, filed on Jul. 2, 2014.

(51) Int. Cl. A61F 7/00

(52) U.S. Cl. CPC A61F 7/007; A61F 2007/0052; A61F 2007/0056; A61F 2007/0093; A61F 2007/0296

(57) ABSTRACT

Devices and methods are presented whereby cryotherapy may be conducted with both an enhanced healing process and a reduced risk of collateral ischemic injury when compared with existing options. A cold effect is applied to an injured tissue for a sufficient time to lower the temperature sufficiently to suppress local pain and inflammation. In an alternating manner a warm effect is applied to the tissue at the same treatment site with sufficient intensity to raise the temperature at the treatment site to equal or exceed the baseline value for a sufficient time to cause an increase in local blood flow equal to or exceeding the baseline value, after which the cycle is repeated until therapy is no longer needed.

### PRIORITY INFORMATION

This application claims priority to U.S. Provisional Patent Application No. 62/020,100, filed on Jul. 2, 2014, and hereby incorporated by reference in its entirety.

### STATEMENT REGARDING FEDERALLY SPONSORED RESEARCH FOR DEVELOPMENT

This invention was made with government support under Grant No. CBET0966998 and Grant No. CBET1250659 awarded by the National Science Foundation and Grant No. R01 EB015522 awarded by the National Institutes of Health. The government has certain rights in the invention.

### BACKGROUND OF THE INVENTION

Field of the Invention

The invention generally relates to devices and methods of cryotherapy. More particularly, the invention relates to devices and methods of cryotherapy that combine alternating heating and cooling of injured tissues to minimize ischemic damage to the tissues and enhance the healing process.

Description of the Background and Relevant Art

Localized cooling is commonly used after orthopedic surgery and in sports medicine to reduce bleeding, inflammation, metabolism, muscle spasm, pain, and swelling following soft tissue trauma and injury. The therapeutic application of cold therapy has a long history dating from the time of Hippocrates and is widely practiced. Over the past two decades the breadth of application has increased dramatically with the advent of mechanized cryotherapy devices consisting of an insulated container filled with an ice/water bath and a submersible pump to propel the flow of ice water through a cooling bladder applied to a therapy site. These devices are now used prescriptively for orthopedic surgical procedures and in many sports and rehabilitation medicine settings. Nonetheless, there remains considerable controversy over the appropriate protocol for application of cryotherapy. One extreme camp advocates continuous use of cryotherapy to a treatment site with no break in cooling for days or even weeks, whereas other practitioners recommend a maximum application duration of 20 to 30 minutes followed by a cessation period of about 2 hours. Many devices and methods are designed and marketed from a perspective that effective and safe cryotherapy depends primarily on regulating the temperature applied to the skin surface, and the duration of cooling is a secondary factor to also be regulated. However, there is a paucity of scientifically derived data that can direct the rigorous and rational design of cryotherapy protocols optimized for therapeutic efficacy and safety. Much of the background understanding that underlies the current practice of cryotherapy is based on anecdotal observations derived from clinical experiences. For example, although continuous cooling appears to be tolerated by many patients, there have been a large number of reported incidences in which continuous application of a cryotherapy device led directly to extensive tissue necrosis and/or nerve injury in the treatment area, sometimes with dire medical consequences.

Although injuries attributed to cryotherapy are frequently classified as frostbite, the fact that cryotherapy units (CTUs) typically use the circulation of melted ice through a flexible pad applied at the treatment site precludes the possibility of actually freezing tissue. Rather, extensive evidence points to tissue damage by nonfreezing cold injury (NFCI) when tissue is subjected to a prolonged state of cold induced vasoconstriction that starves tissues of oxygen and nutrients and allows the accumulation of toxic metabolic byproducts. Therefore, although an applied low temperature is the factor that defines cryotherapy and precipitates both the beneficial and damaging tissue responses, a strong case can be made that the most expedient approach to the design of cryotherapy devices and methods should be guided by considerations of controlling how the perfusion of blood to the treatment area should be manipulated over time.

It is well known that a lowered tissue temperature depresses the conduction velocity in nerves, which, in combination with local ischemia, is thought to relate to the incidence of nerve injury. Cell necrosis may result from a number of complicating factors precipitated by cold-induced ischemia. It has been known for many years that reduced temperatures cause a local decrease in blood perfusion of advantage in treating soft tissue injuries by limiting swelling and inflammation. But, when a prolonged state of ischemia is maintained, cells are deprived of a sufficient supply of nutrients in conjunction with the buildup of metabolic byproducts that, taken together, may lead directly to tissue necrosis and neuropathies. Causation of a prolonged state of ischemia also can lead to the occurrence of reperfusion injury when blood flow is reestablished to the affected tissue. In some cases, these types of injuries are the unfortunate byproduct of the application of cryotherapy. Thus, there is a need to achieve a balance between deriving the benefits of applied cryotherapy while reducing the risk of causing further injury to the tissue being treated, especially when an inherent, concurrent outcome of applying the disclosed invention is an additional improvement in tissue healing.

### SUMMARY OF THE INVENTION

Devices and methods are presented whereby cryotherapy may be conducted with both an enhanced healing process and a reduced risk of collateral ischemic injury when compared with existing options. Modulation of the applied therapeutic temperature over time is used to achieve the dual objectives of improved healing processes and lower risk of collateral injury.

Avoiding long term ischemia during cryotherapy of extended duration may be achieved by an intermittent raising of the tissue temperature to transiently increase perfusion. For this purpose it is desirable to alternate the skin temperature between lower (15° C.-20° C.) cooling values and higher (37° C.-42° C.) warming values. Cooling allows the following therapeutic efficacies to be achieved: (1) to lower blood perfusion for reduced tissue swelling; (2) to lower nerve conduction velocity for reduced pain sensation; and (3) to reduce inflammation processes.The short periods of heating allow: (1) elevation of blood flow and metabolic rates to avoid long term ischemia and the potential for tissue injury; (2) prevention of subsequent ischemic reperfusion injury; and (3) improved rates of tissue recovery by exposing the tissue to occasional warm temperatures where healing biochemical processes can proceed at a normal rate. In one embodiment, the duty cycle for intermittently warming tissue from the cryotherapeutic state would be on the order of 5% to 10% of the total therapy time. In another embodiment it may be on the order of 35% to 40%. This ratio may be varied independently over a broad range of states to address a variety of medical and therapeutic applications. The desired frequency of the warming episodes may be adjusted to meet the needs of a patient, although, in some embodiments, the longest period of continuous cooling should not exceed beyond 60 minutes; in some embodiments, the longest period of continuous cooling should not exceed beyond 30 minutes. The method described herein has the added benefit of providing a brief period of higher metabolism to contribute to improved healing rates and overall tissue vitality.

The present method is designed to not allow the development of a state of persistent ischemia in the treatment area. It may be adopted for application with existing technologies or may be implemented with a more sophisticated and accurate means of skin temperature control with the objective of preventing reduced blood flow or to stimulate blood flow. The present invention may further embody a thermal barrier designed for explicit regulation of the skin temperature, and in each event retains the benefits of the practice of cryotherapy.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1A depicts an embodiment of a cryotherapy device;

FIG. 1B depicts an additional view of the cryotherapy device of FIG. 1A;

FIG. 2 depicts a schematic diagram of an alternate embodiment of a cryotherapy device;

FIG. 3 depicts a schematic diagram of a third embodiment of a cryotherapy device;

FIG. 4 depicts a schematic diagram of a control system for operating a cryotherapy device;

FIG. 5 depicts thermal stimulation of blood flow by active warming with an electric heater placed onto the surface of a cryotherapy pad, alternating with active cooling via circulating cold water;

FIG. 6 depicts thermal stimulation of skin blood flow during cycles of active cooling alternating with active warming;

FIG. 7 depicts thermal stimulation of skin blood flow during increasingly longer cycles of active cooling alternating with active warming;

FIG. 8 depicts thermal stimulation of skin blood flow during cycles of active cooling, using circulating cold water, and alternating with active warming using an electric heater;

FIG. 9 depicts thermal stimulation of skin blood flow during cycles of active cooling alternating with active warming using thermoelectric modules;

FIG. 10A depicts a computer simulation of the transient temperature cycling and penetration within the underlying skin in response to alternating cooling and warming with one cycle consisting of minutes surface cooling at 15° C. and 7.5 minutes warming at 40° C.;

FIG. 10B depicts a computer simulation of the transient temperature cycling and penetration within the underlying skin in response to alternating cooling and warming with one cycle consisting of 20 minutes surface cooling at 15° C. and 3 minutes warming at 40° C.;

FIG. 11 shows recorded temperature output from a thermoelectric module regulated by an H-bridge controller to produce a sine wave pattern to apply for therapeutic application;

FIGS.12A-D show alternative arrangements of multiple thermoelectric modules to achieve enhanced thermal performance and/or to generate more than a single controlled temperature fluid stream that may be used for therapeutic purposes; and

FIG. 13 depicts the data flow of a programmable temperature controller interfaced with a thermoelectric module and having a user interface.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

The term “cryotherapy” is defined as local therapeutic cooling of tissue.

The term “temperature control device” is defined as a system or apparatus with means for regulating the temperature applied to a body part of a mammal for a treatment or for obtaining a therapeutic outcome.

The term “cooling source” is defined as a system or apparatus with means for reducing the temperature of tissue in a region to which it is applied.

The term “warming source” is defined as a system or apparatus with means for increasing the temperature of tissue in a region to which it is applied.

The term “programmable controller” is defined as a system or apparatus that enables a user, such as a health care provider, to input a targeted temperature time history that a thermal therapy device will execute onto a tissue region.

The term “tissue temperature monitoring device” is defined as one or more sensor transducers that can be applied to the surface of the skin to continuously measure the temperature at a sensor location and to provide a feedback signal to a temperature control device that is operating to execute a programmed temperature time history in a tissue region.

The term “blood flow stimulation device” is defined as any means for causing the blood flow to increase in an area of tissue on demand, and, in particular, to increase from an ischemic or vasoconstricted state to a normal state and to return to an ischemic or vasoconstricted state when the stimulation is withdrawn. In the present invention the stimulation of blood flow may include primarily the application of a controlled heating effect to the tissue, although it may be combined with other stimulation means including, but not be limited to, the following methods: manual massage; device massage; device vibration; transient flexing of muscles; physical movement of a body part; ambulation; application of Transcutaneous Electrical Nerve Stimulation (TENS); application of Electrical Muscle Stimulation (EMS); altering the elevation of a body part; standing, walking, jumping on an elastic surface; application of impulse blood pump stimulation; application of a sequential pneumatic compression blood pump distally, locally, or proximally; application of roller stimulation; application of active and/or passive flexion and extension on proximal and/or local and/or distal joints; stimulation distal, local and/or proximal to the cryotherapy site; local heating of tissue at the treatment site, which is the preferred means in this application; any other method known to potentially increase local blood flow.

The term “heat transfer limiting thermal barrier” is defined as a material positioned between a temperature control device and the portion of the tissue to cause a temperature drop between these two elements to thereby limit the rate of heat exchange with the portion of tissue.

The term “treatment temperature” is defined as the temperature applied to a tissue or treatment area to produce a therapeutic outcome. In the present invention the treatment temperature may vary with time according to a predetermined pattern.

The term “cooling and warming cycle” is defined as a process in which the tissue at a treatment site is cooled under defined temperature and time conditions, following which it is warmed under independently defined temperature and time conditions, subsequent to which the process may be repeated cyclically as may be required.

The term “thermoelectric module” is defined as a solid state device to which an electric voltage may be applied with a given magnitude and polarity to produce a temperature differential across its surfaces, one side of which is at a temperature below the ambient value, which temperature differential is inverted when the voltage polarity is reversed.

The term “cooling/warming ratio” is defined as the ratio of times for which active cooling and warming are applied to a tissue treatment site during a complete cooling and warming cycle. When more than a single cycle is applied, the ratio may vary for different cycles.

The term “time constant” is defined as the time that a temperature is applied to a tissue or treatment area to produce a therapeutic outcome. The time constants for cooling and warming may be different and may vary over the total course of a treatment protocol.

The term “preconditioning” is defined to broadly encompass conditioning of tissue before a particular injury or damage occurs, in order to improve recovery of the tissue after the injury or damage. Preconditioning may include brief episodes of high or low temperature alternatively applied to a treatment tissue to upregulate the expression of heat shock proteins in cells to provide an enhanced healing process and resistance to ischemic injury.

Methods described herein are used with associated devices to solve the problem caused by cryotherapy producing a state of persistent and profound ischemia that can lead to injury causation via tissue necrosis and neuropathy. The method mitigates the injury causation while retaining the therapeutically beneficial advantages of the cold treatment. Cryotherapy is thought to provide a favorable affect to injured tissues by pain analgesia and diminished swelling and inflammation. The latter may result from cold induced vasoconstriction that issues in reduced fluid extravasation in conjunction with a diminished rate and volume of blood flowing to tissue in the treatment area. Thus, it is thought to be important that, for a net majority of the cryotherapy treatment time, it is desirable for the tissue temperature and blood perfusion rate both to be reduced below normal values. However, in order to avoid the progressive buildup of the conditions conducive to ischemic injury, it is necessary to create intermittent periods during which the tissues in the treatment area are perfused with a supply of fresh blood to provide the oxygen and nutrients requisite to support cell metabolism and to wash out accumulated metabolic by products in order to avoid cell death. In addition, a periodic perfusion with fresh blood will be conducive to the healing process, especially if it occurs simultaneously with a rise in tissue temperature. Given that cryotherapy is often prescribed for continuous use durations measured in consecutive days or longer, being able to maintain the foregoing balance is all the more critical to effective and safe cryotherapy. All embodiments set forth herein meet this set of criteria.

In order to function for its intended purpose, the present invention must be able to overcome a deep state of vasoconstriction and persisting ischemia induced by cryotherapy on demand to return the blood flow to tissues to normal levels for a measured period of time that can be specified independently, and then to return the flow to the prior depressed levels for the sake of the cryotherapy efficacy. It will be shown from our human subject data that all of the present embodiments are fully functional according to this principle.

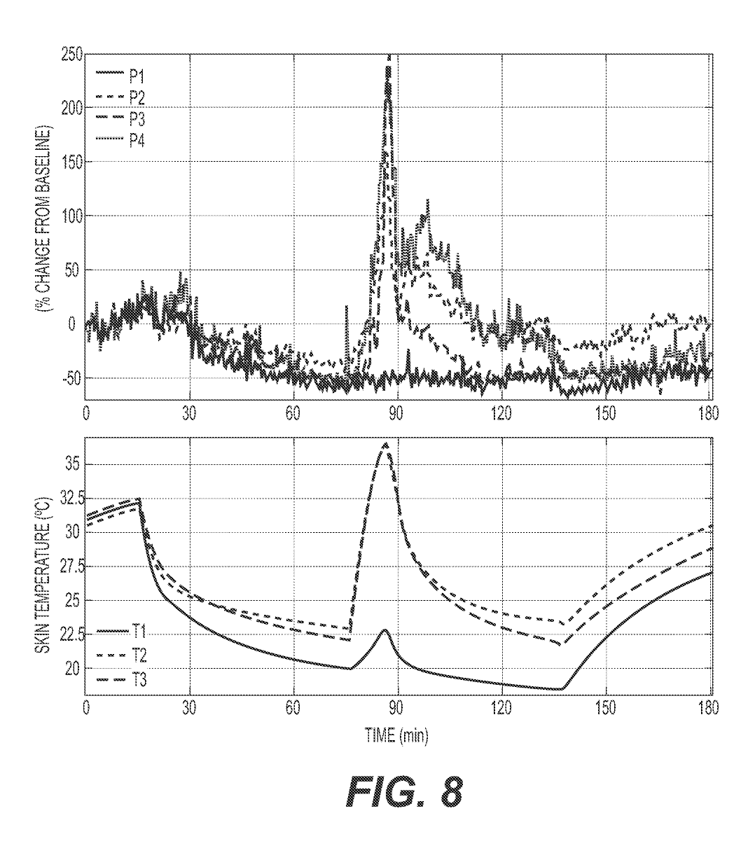

With respect to the cyclic cooling and warming of the tissue in the treatment area, this method consists of cooling the tissue for cryotherapy followed by active warming of the tissue for a defined period of time and then resumption of cooling to initiate another cycle. Example protocols are illustrated in FIGS. 6, 7, 8, and 9. The controlled process is the temperature applied to the skin surface. The physiological response is characterized in terms of the blood perfusion in the treatment area. Note that during active cooling the perfusion drops rapidly. Likewise, during active warming, the perfusion rises rapidly. Conversely, during passive cooling and warming the perfusion remains in a largely static condition. Therefore, this method requires intervention with active cooling and warming to rapidly drive the local blood perfusion between high and low levels to achieve the cryotherapy effect and protection against ischemic injury. An important feature of this method is that the active warming to overcome the cold-induced ischemic state does not block the subsequent return to deep vasoconstriction with the resumption of active cooling. Thus, the balance between cryotherapy effectiveness and injury prevention is achieved.

In one embodiment, an apparatus that provides cooling and mechanical and/or electrical simulation to the treated site may be used to overcome the problems associated with cryotherapy. In one embodiment, a cryotherapy device for producing a cooling effect for treating tissue includes a temperature control device which alters the temperature of at least a portion of the tissue being treated during treatment; and a blood flow device that alters the blood flow rate through the portion of the tissue being treated during and following treatment.

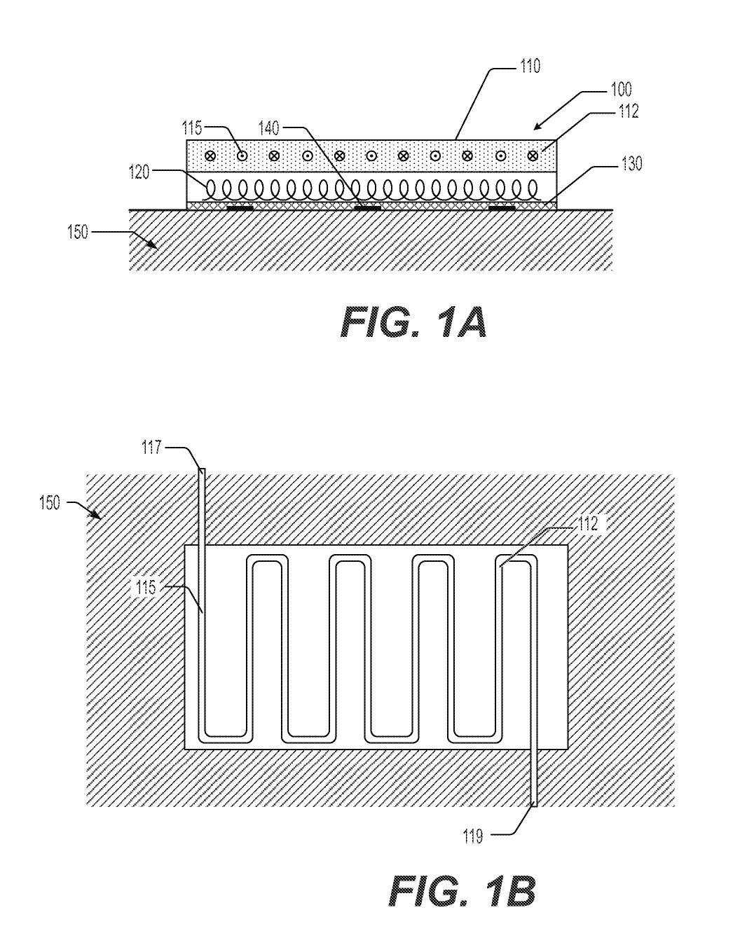

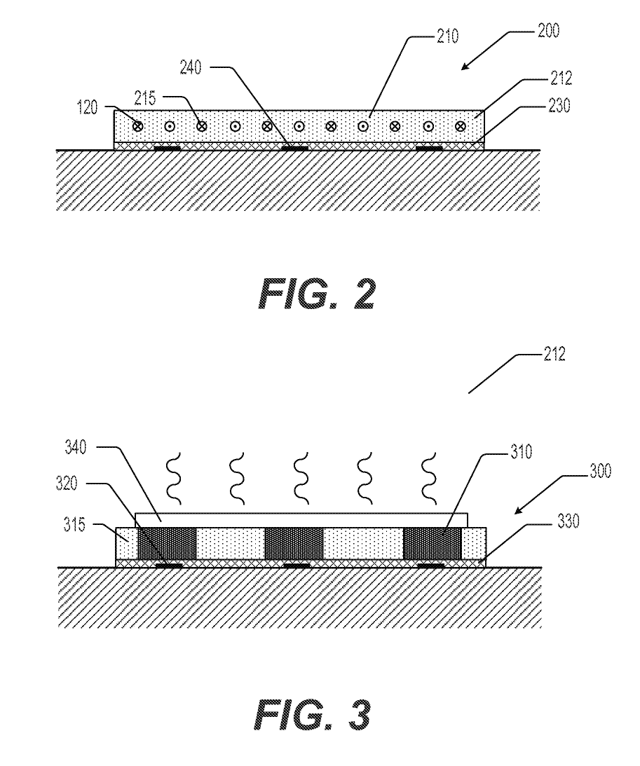

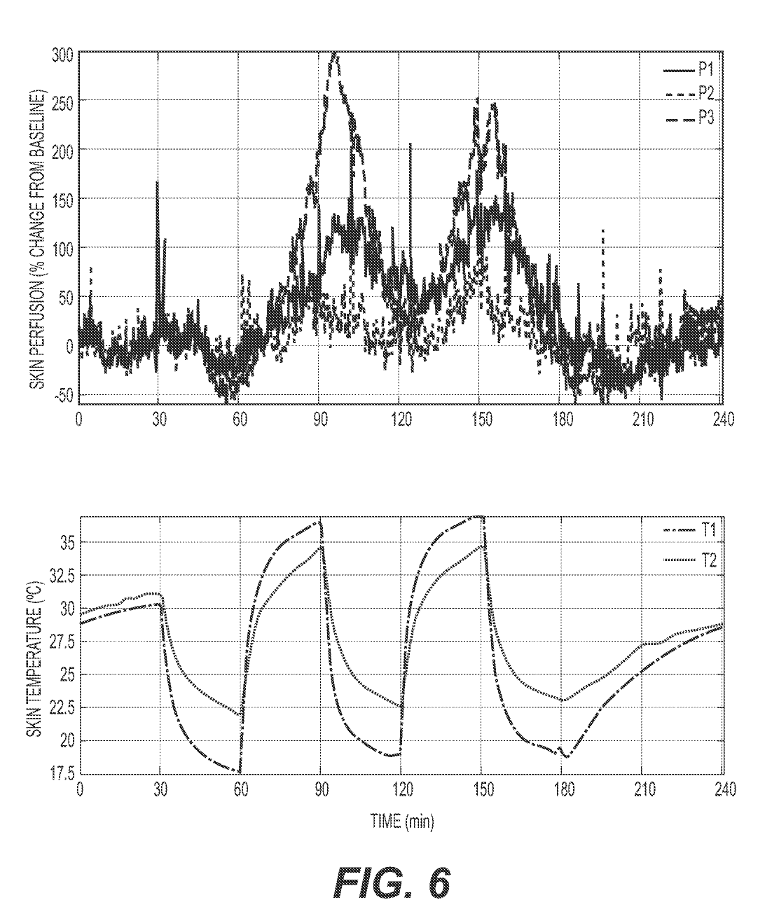

FIGS. 1A-B depict an embodiment of a cryotherapy device 100. Cryotherapy device 100 includes a liquid circulation bladder 110, an electric heating device 120, and a thermal barrier 130, arranged as depicted in FIG. 1. Liquid circulation bladder 110 includes a support medium 112 and a serpentine conduit 115 which extends throughout the support medium. Serpentine conduit 115 includes an inlet 117 and an outlet 119 which transmit cooling fluid from an external source of cooling fluid (not shown) through the serpentine conduit. One or more tissue temperature sensors 140 are used for feedback to a programmable controller (see FIG. 4). The device shown is incorporated into a single unit that is applied at a site of therapy. The device is positionable on the tissue 150 of a subject.

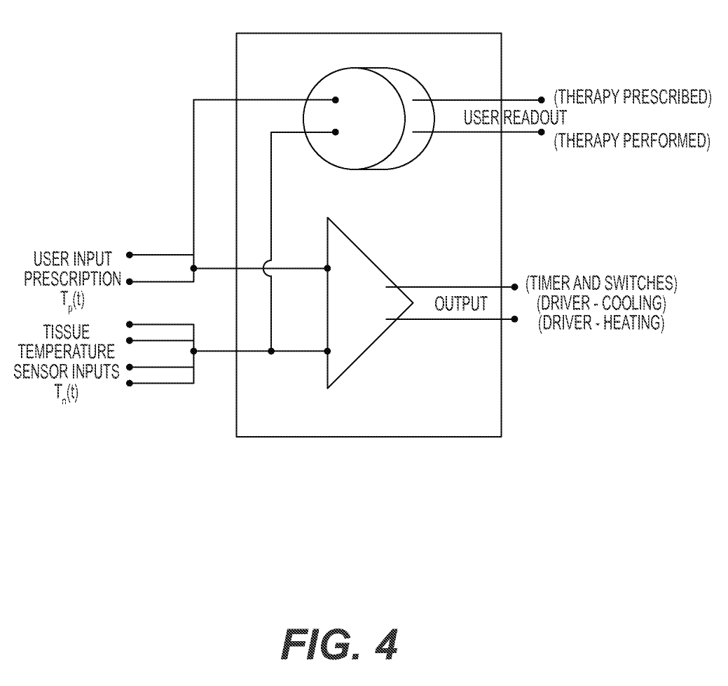

FIG. 2 depicts an embodiment of a cryotherapy device 200. Cryotherapy device 200 includes a liquid circulation bladder 210 and a thermal barrier 230, arranged as depicted in FIG. 2. Liquid circulation bladder 210 includes a support medium 212 and a serpentine conduit 215 which extends throughout the support medium. Serpentine conduit 215 includes an inlet and an outlet which receive cooling fluid and heating fluid from an external source (not shown). One or more tissue temperature sensors 240 are used for feedback to a programmable controller (see FIG. 4). The device shown is incorporated into a single unit that is applied at a site of therapy

In one embodiment the temperature control device includes a heat transfer substrate which receives a cooling fluid during treatment of the portion of the tissue and transfers heat between the cooling fluid and the tissue. In an alternate embodiment, the temperature control device is composed of a thermoelectric material, and wherein the temperature of the thermoelectric material is altered by applying a voltage to the thermoelectric material. The cryotherapy device may also include a voltage source capable of supplying alternating applied voltage to thermoelectric material to create alternate heating and cooling of the portion of the tissue.

In an alternate embodiment, the temperature control device includes multiple segments that include a thermoelectric material. Each of the segments may be controlled individually, the segments being mounted on a flexible substrate that is capable of conforming to the surface geometry of the portion of the tissue. The multiple thermoelectric segments may be arranged and controlled so as to produce a temperature grid of cooler and warmer regions on the portion of the tissue that can be modulated in both position and time so as to produce a desired therapeutic cooling effect with reduced risk of causing induced injury to the treatment and adjacent areas.

In another embodiment, a device and method may combine active warming of the treatment area with active cooling. The combination of warming and cooling may be modulated in combinations of spatial and temporal patterns. The cooling and warming may be produced by a matrix of thermoelectric devices in thermal contact with the skin at a treatment area that can be modulated in patterns that vary in space and/or in time to produce a desired temperature effect and blood perfusion effect. The devices and methods may incorporate a thermal sensor in the area of therapy and a blood flow sensor in the area of therapy, both of which may be connected to a control function to regulate the cryotherapy system to achieve cooling benefit while avoiding unnecessary risk of causing ischemic injury.

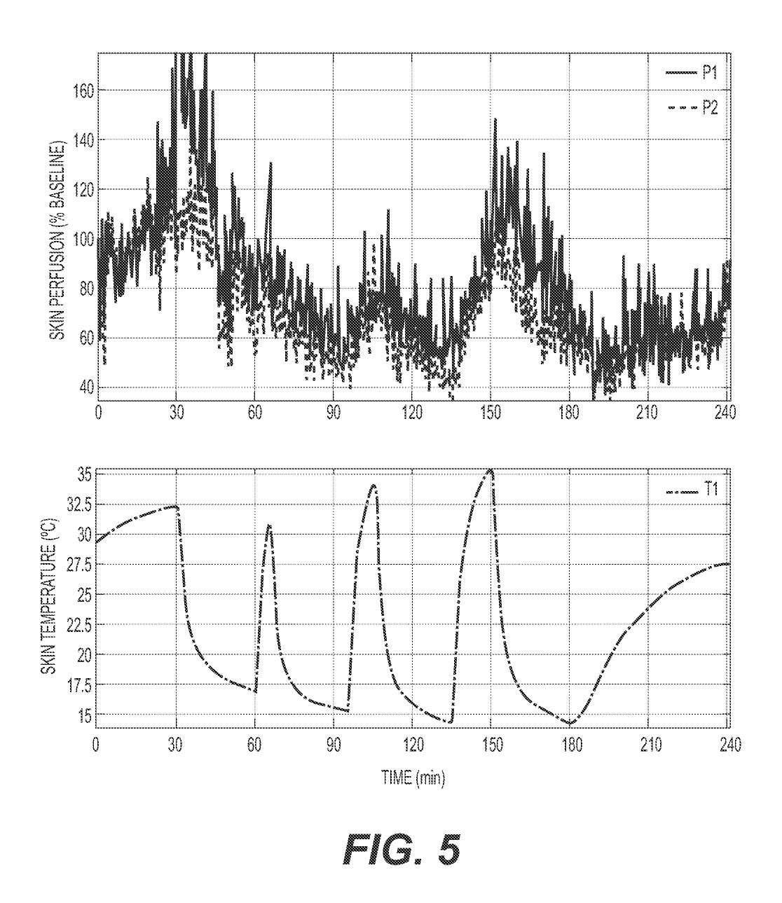

In FIG. 3, an alternate embodiment of a cryotherapy device 300 is shown. Cryotherapy device 300 includes a matrix of individually controlled thermoelectric modules 310, a tissue temperature sensor 320 for each module, a passive thermal barrier 330, and an elastic mounting material 315 to hold the modules in conformity to the tissue anatomy. Forced convection cooling of the hot side of the modules (e.g., using fan 340) is used to help regulate the temperature of thermoelectric modules. A ducting may be provided to control the flow of air directed from the fan. The ducting may be flexible and deformable to match local tissue anatomy and to protect against contact with the high temperature side for the thermoelectric material. Each thermoelectric module 310 is independently controlled through independent inputs from a programmable controller. This embodiment uses a thermoelectric material placed on the site of therapy that can alternatively apply cooling and heating of specified strengths by regulating the control voltage. This embodiment has multiple advantages of: eliminating a remote source of cooling and the requirement of circulating a chilling solution between the source and treatment site; combining cooling and warming functions into a single device; reduced size and mass; lower operating sound; reduced refrigeration loss; higher thermodynamic efficiency during cooling and heating cycles; reduced power requirements.

FIG. 4 depicts a control system for regulating the alternating cooling and heating of tissue during cryotherapy. The control system includes a user input for programming a prescribed temperature history to be applied to tissue to produce a desired temperature and blood perfusion history. Continuous tissue temperature inputs from the treatment site are used to record temperature histories at defined sites on the tissue at the active treatment site. Data storage is provided to allow storage and downloading of the treatment history for documentation to health care providers. The control system is run using a PID logic processer to generate an output control signal to regulate the timing and magnitude of cooling and warming effects at the tissue therapy site.

The cryotherapy device may include a variety of sensors to monitor the conditions of the tissue being treated. For example, the cryotherapy device may include: one or more temperature sensors coupleable to the tissue to measure the surface temperature of the tissue; one or more blood flow rate sensors coupleable to the portion of the tissue to measure blood perfusion in the tissue; and one or more oxygenation sensors coupleable to the portion of the tissue to measure the level of oxygenation in the tissue. The cryotherapy device may also include a controller coupled to one or more of the temperature sensors, one or more of the blood flow rate sensors and one or more of the oxygenation sensors. The controller may use the data collected from one or more of the sensors to modulate the operation of the temperature control device and the blood flow device to achieve a desired therapeutic outcome.

In another embodiment a device and method for applying cooling to the skin surface comprises a time/temperature history which effectively limits the extent of blood flow depression in the treatment area and distal areas.

Although several embodiments discussed herein refer to treating tissue in the context of recovering from injury, all of the embodiments discussed herein may also be applied to preconditioning tissue prior to injury. For example, preconditioning can be particularly useful prior to surgery in order to prepare the tissue for injury by upregulating heat-shock proteins that aid in the repair of molecular damage. Preconditioning may involve, for example, upregulating these heat-shock proteins for an extended time prior to surgery so that the elevated concentration still exists at the time of surgery.

Generally, preconditioning occurs by applying a sub-lethal stress to cells for a sufficient period of time to cause an upregulation of heat-shock proteins that subsequently lead to an improved healing process. Preconditioning may be accomplished by a variety of types of stress, including heating, cooling, radiation, and chemical means. However, exceeding a certain heating threshold may kill the cells, while cooling for beyond a certain time threshold can cause ischemic injury

Preconditioning may involve heating the cells over time. However, temperature-contrast therapy may also be used for preconditioning. For example, a thermoelectric source capable of alternatively heating and cooling may be used to accomplish the preconditioning. In one embodiment, preconditioning by heating may be accomplished by heating for about 3 or more hours without any cooling. Longer durations or time may provide additional benefits, although the cells should be kept below a lethal level of heat. For example, the cells should be kept below about 43° C.

In the case of preconditioning via contrast therapy to strengthen the upregulation of heat-shock proteins while avoiding causing lethal cell injury, a variety of transient temperature and time protocols could be applied. For example, the temperature may be varied between a cold temperature, such as 15° C., and a warm temperature, such as 43° C. Other temperatures may be used as well, depending on the particular needs of the patient. For example, cold-temperature ranges between 10-20° C. may be used, while warm-temperature ranges between 30-45° C. may be used. Additional temperatures are also possible to use with some positive effect.

The duration of the contrast therapy may also vary based on the patient's needs. In one embodiment, alternating 3-hour periods of time are used. In another embodiment, alternating 1.5-hour period of time are used. Any suitable time periods may be used—for example, the time periods may range from about 1 hour to over 10 hours. In addition, the heating and cooling periods may be different. For example, the preconditioning may use 3-hour periods for warming and 2-hour periods for cooling. Furthermore, the time periods may change as the contrast therapy progresses. An example would be using 3-hour time periods for a single round of heating and cooling, followed by 1.5-hour time periods for the next round of heating and cooling, and so on.

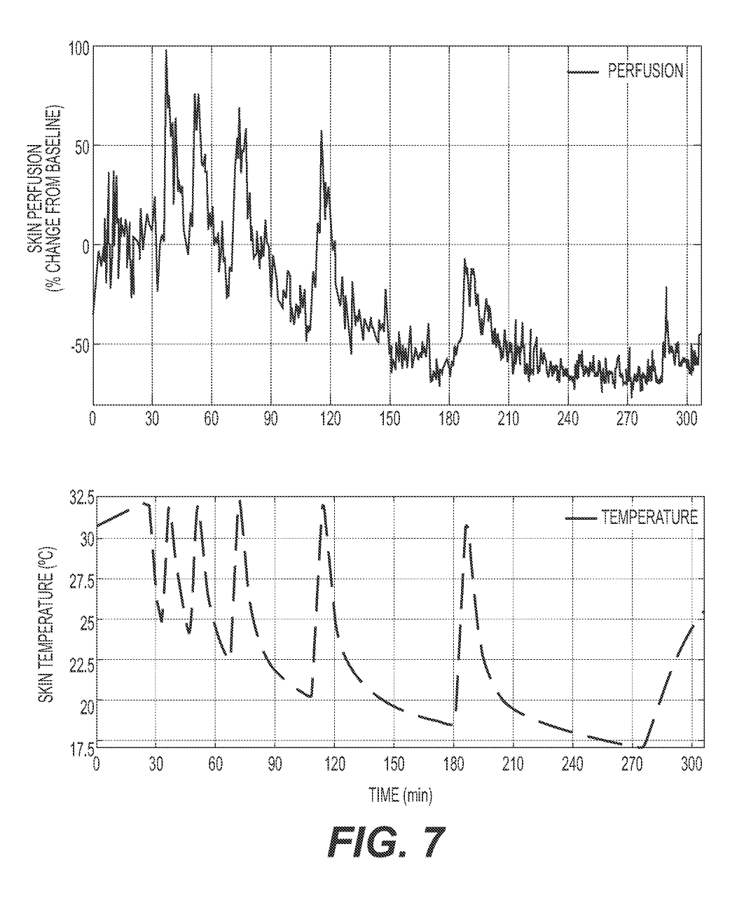

FIG. 5 depicts thermal stimulation of blood flow by active warming with an electric heater placed onto the surface of a cryotherapy pad, alternating with active cooling via circulating cold water. Active cooling is performed for 30 minutes. Three periods of active warming last progressively for 5 minutes, 10 minutes and 15 minutes.

FIG. 6 depicts thermal stimulation of skin blood flow during cycles of active cooling alternating with active warming. Active warming is accomplished with an electric heater placed onto the surface of a cryotherapy pad. Active cooling is accomplished with circulating cold water. 30 minutes of active cooling is followed by 30 minutes of active warming. Two full cycles are shown, followed by a single cooling episode after which the system was shut off allowing passive rewarming to progress.

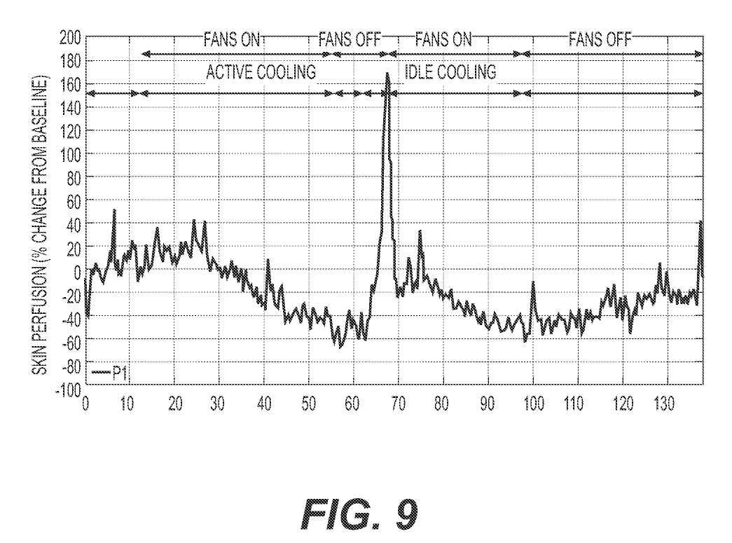

FIG. 7 depicts thermal stimulation of skin blood flow during increasingly longer cycles of active cooling alternating with active warming. In this example, blood flow was stimulated by 5 minutes of active warming with an electric heater placed onto the surface of a cryotherapy pad, alternating with active cooling via circulating cold water for progressively increasing durations for 5, 10, 15, 30, 60, and 90 minutes, after which the system was shut off allowing passive rewarming to progress.

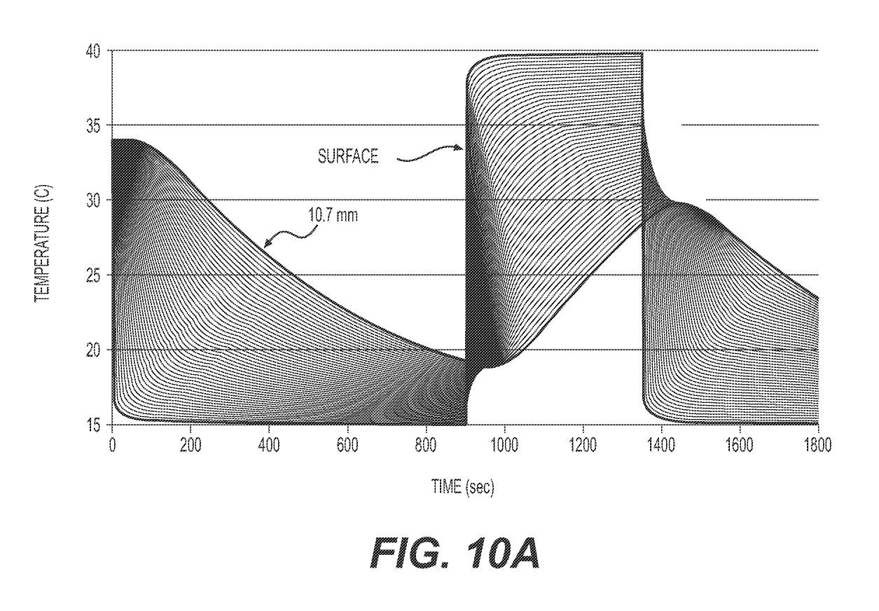

FIG. 8 depicts thermal stimulation of skin blood flow during cycles of active cooling, using circulating cold water, and alternating with active warming using an electric heater. All perfusion and temperature probes were positioned under the cooling pad, but P1 and T1 were not located under to heater to serve as a control to characterize the effect of active warming on perfusion and temperature.

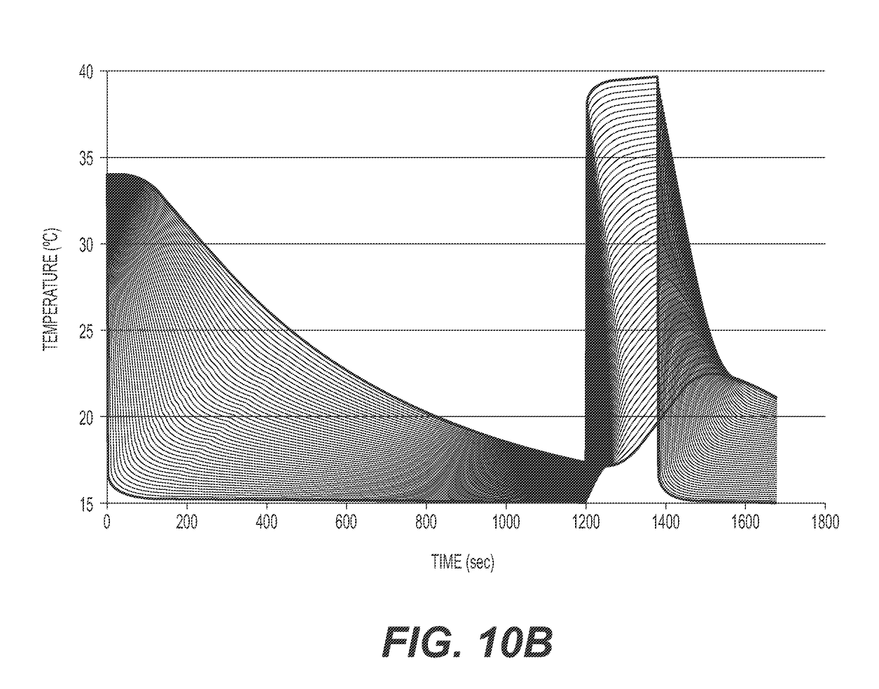

FIG. 9 depicts thermal stimulation of skin blood flow during cycles of active cooling alternating with active warming using thermoelectric modules. Thermal stimulation of blood flow during cryotherapy was performed by controlled modulation of multiple thermoelectric modules applied to the skin over a temperature distribution barrier. Following 25 minutes of baseline data with the thermoelectric modules in place and their cooling fans operating the final 10 minutes, active cooling by direct thermoelectric refrigeration occurred for 30 minutes followed by 5 minutes of passive warming with the entire system off, then by 5 minutes of active thermoelectric warming, then 30 minutes of thermoelectric refrigeration, and finally 40 minutes of passive rewarming with the entire system turned off but still in position on the skin. Skin blood perfusion was monitored between adjacent thermoelectric modules.

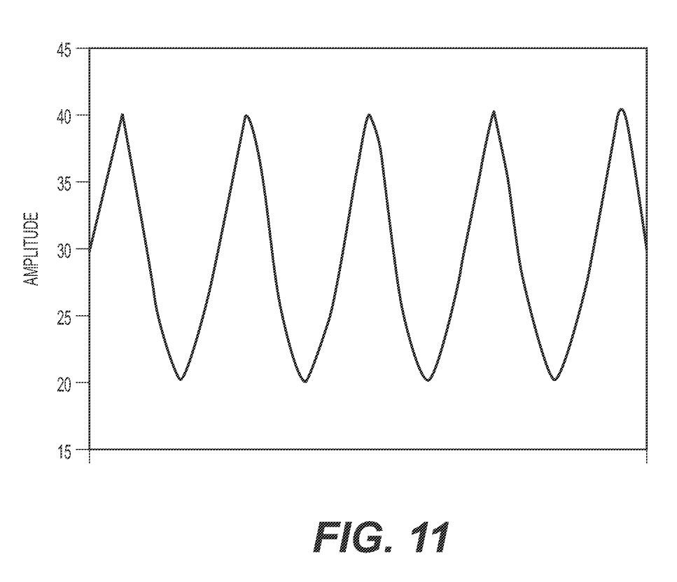

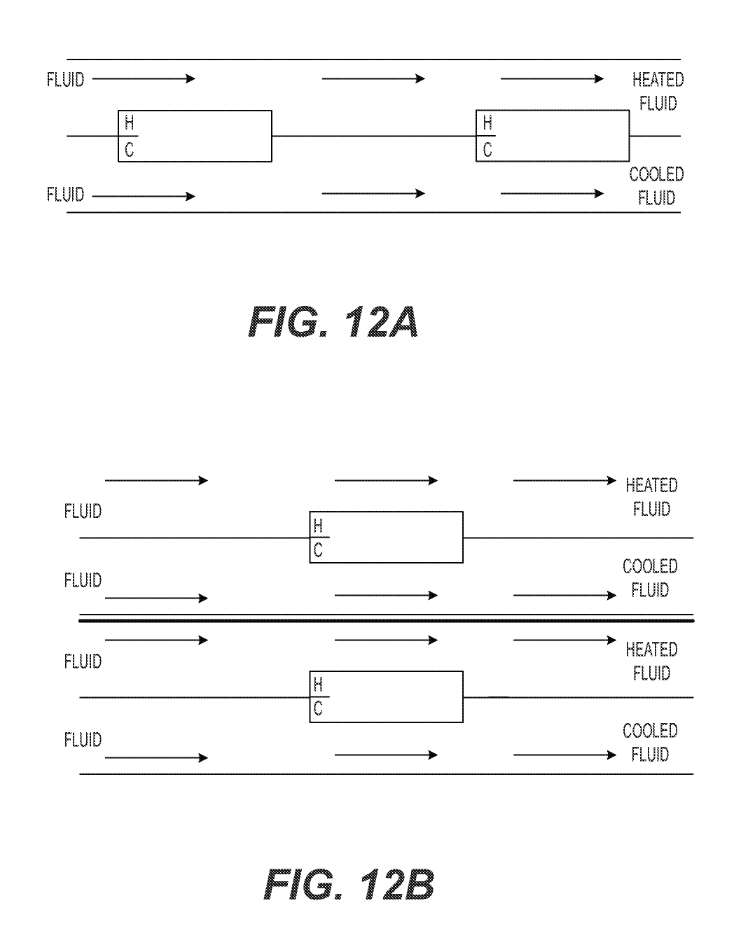

FIGS. 10A and B depict computer simulations of the transient temperature cycling and penetration within the underlying skin in response to alternating cooling and warming. In FIG. 10A the cycle used cooling for 15 minutes and warming for 7.5 minutes applied on the surface.

In FIG. 10B the cycle used cooling for 20 minutes and warming for 3 minutes applied on the surface.

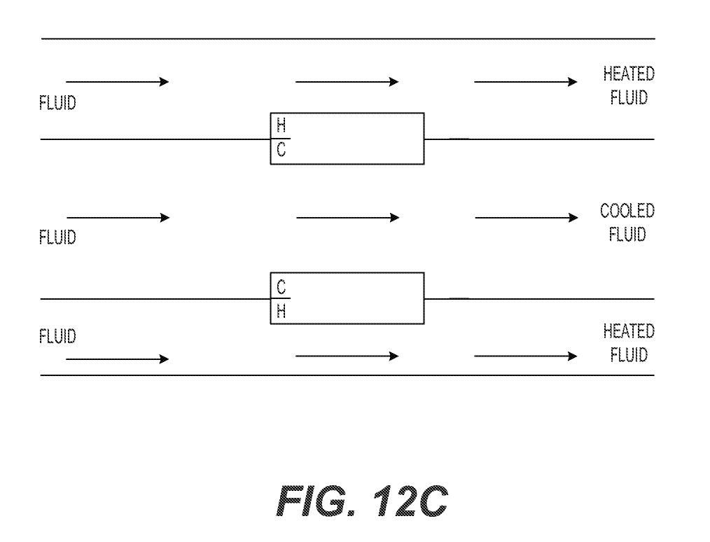

FIG. 11 shows recorded temperature output from a thermoelectric module regulated by an H-bridge controller to produce a sine wave pattern to apply for therapeutic application.

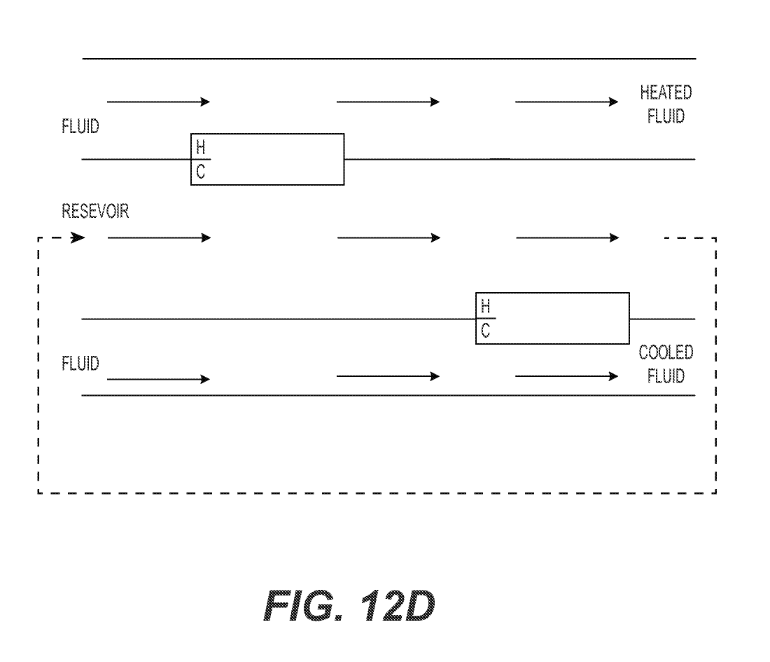

FIG. 12 shows alternative arrangements of multiple thermoelectric modules to achieve enhanced thermal performance and/or to generate more than a single controlled temperature fluid stream that may be used for therapeutic purposes. FIG. 12A depicts two modules in series that share common hot and cold fluid streams. FIG. 12B depicts two modules in parallel with separate hot and cold fluid streams. FIG. 12C depicts two modules in parallel with a common cold fluid stream and separate hot fluid streams. FIG. 12D depicts two modules in series with separate hot and cold fluid streams and a common working reservoir fluid stream that exchanges with the cold side of one module and then the hot side of the other module and is recirculated within the array of modules.

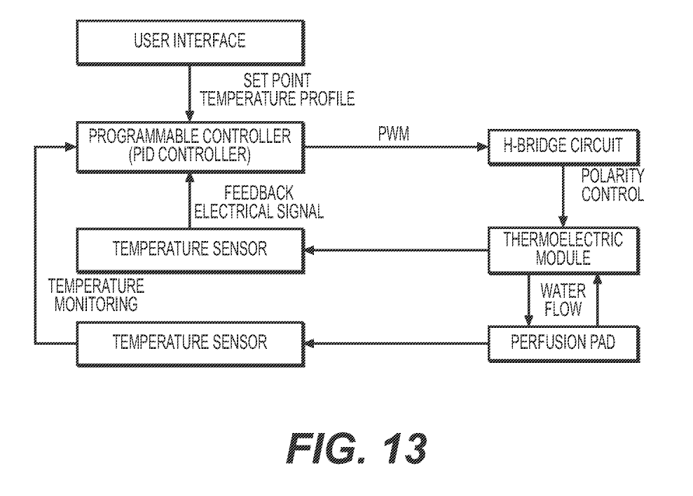

FIG. 13 shows the data flow of a programmable temperature controller interfaced with thermoelectric module and having a user interface. PWM=pulse width modulation. PID=proportional integral derivative.

The results demonstrate that even short episodes of cryotherapy can produced a prolonged duration of ischemia in the local area of treatment as well as in more distal tissues. The state of ischemia endures long after local temperatures have rewarmed toward the normal range, indicating the ischemia is not directly coupled to a cold state. It is likely that a long acting humoral vasoconstrictive agent is released locally that continues to be active long after tissue temperatures have rewarmed toward their baseline values. Evidence for the action of such vasoconstrictive agents has been confirmed in the occurrence of nonfreezing cold injury. The suppression of blood perfusion at sites distal to the site of applied cryotherapy may be a consequence of the vasoconstrictive agent eventually washing downstream in the residual blood flow, causing distal vasoconstriction in tissues that were not affected directly by the cryotherapy.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A method for improving tissue healing and for preventing collateral ischemic injury during cryotherapy comprising:
thermally coupling a cryotherapy device to a portion of the tissue, wherein the cryotherapy device comprises a cooling component and a warming component;
operating the cooling component of the cryotherapy device to cool a portion of the tissue; and
operating the warming component of the cryotherapy device to actively warm the portion of the tissue during a time period when it is not being cooled.

2. The method of claim 1, wherein the cryotherapy device is operated cyclically in pairs of cooling and warming phases.

3. (canceled)

4. The method of claim 1, wherein the cooling treatment temperature applied to the skin is set to be above values that produce substantial ischemia in the tissues.

5. The method of claim 1, wherein the cooling treatment temperature is selected such that the temperature of the tissue is in the range of 16° C.-26° C.

6. The method of claim 1, wherein the cooling treatment temperature is selected such that the cooling temperature of the tissue is in the range of 12° C.-15° C.

7. The method of claim 1, wherein the warming treatment temperature is selected such that the temperature of the tissue is in the range of 37° C.-42° C.

8. The method of claim 1, wherein the warming treatment temperature applied to the skin is set to not be higher than a value that produces irreversible thermal injury to the tissues.

9. (canceled)

10. The method of claim 1, wherein the therapeutic cooling and warming cycle may be repeated for multiple cycles.

11. The method of claim 2, wherein the ratio of the durations of the cooling and warming components of a cycle may be changed for sequential cycles according to a predetermined pattern.

12. (canceled)

13. (canceled)

14. A device for applying cryotherapy to a region of a mammal without causing a state of prolonged ischemia and to periodically increase the temperature and the rate of blood flow through tissue in the application region, comprising:
a tissue temperature monitoring device;
a thermal barrier device; and
a temperature control device that incorporates both a cooling source and a warming source;
wherein the tissue temperature monitoring device can sense and transduce the temperature at one or more locations in a tissue region and provide one or more signals to a temperature control device to regulate the administration of a thermal therapy in the tissue region;
wherein the temperature control device that modulates the temperature of the cryotherapy device to a designated value that may vary over time based on a feedback signal;
wherein the thermal barrier device has designated heat conduction properties that can regulate the temperature drop between the cooling source and the warming source in the cryotherapy device and the skin;
wherein the temperature control device can be operated periodically according to a pattern designated by a user to produce alternating patterns of low temperature and reduced blood flow and of elevated temperature and increased blood flow in a tissue treatment region; and
wherein the low temperature causes a decrease in blood flow to tissue during cooling to provide the benefits of cryotherapy and the elevated temperature causes an increase in blood flow to tissue to provide a fresh supply of oxygen and nutrients in a state in which the availability of oxygen in blood is higher which promotes an accelerated healing rate.

15. The device of claim 14 wherein the temperature control device causes a cooling effect either by a convective flow of refrigerated liquid originating from a remote source or by one or more thermoelectric cooling modules that operate directly at the treatment site without the use of an intermediate circulating liquid.

16. The device of claim 15 wherein the temperature control device causes a warming effect either by a convective flow of warmed liquid or by a thermoelectric warming module that operates directly at the treatment site without the use of an intermediate circulating liquid or by a flexible electrical heater affixed directly to the surface of a flexible circulating liquid bladder.

17. The device of claim 16 wherein the cooling effect and the warming effect are incorporated into a single physical unit that can be used alternatingly to produce cooling and warming in a tissue region.

18. The device of claim 17 wherein the cooling effect and the warming effect operate at different times that are regulated by the temperature control device under that action of a programmable controller to produce a prescribed cooling/warming ratio.

19. The device of claim 18 further comprising a user input that allows a user to input prescribed temperature time histories during one or more cooling and warming cycles into a programmable controller that will be used by a temperature control device to produce a desired thermal therapeutic effect in a tissue region.

20. The device of claim 19 wherein the temperature monitoring device comprises one or more inert sensors that are integrated into the thermal barrier so as to be placed onto the surface of a tissue region and provide one or more input signals to a programmable controller coupled to the temperature control device.

21. The device of claim 19 wherein the programmable controller receives input signals from the temperature monitoring device and prescriptive control signals from a user and logs and stores this information as time series data that may be accessed by the user at a later time for review of the thermal therapy that was prescribed for and executed by the cryotherapy device.

22. The device of claim 15 wherein the cooling effect is accomplished by a stream of cooled liquid from a remote cold source passing through a flexible bladder applied to a tissue treatment region and the warming effect is accomplished by energizing a flexible electrical heater applied to the heat transfer surface of the flexible bladder.

23. The device of claim 16 wherein the cooling effect is accomplished by one or more thermoelectric modules arranged to conform to the surface of a tissue treatment region and operated to produce a controlled refrigeration effect on the module active side oriented toward the tissue and the warming effect is accomplished by the same one or more thermoelectric modules but with the voltage polarity reversed to produce a warming effect on the active side oriented to the tissue.

24. (canceled)

25. (canceled)

26. The device of claim 18 wherein the cycling between the cooling effect and the warming effect occurs as comprising:
a cooling effect that produces a temperature that is lower than ambient air temperature and a warming effect that is higher than ambient air temperature;
a transition between cooling and warming that occurs continuously and smoothly without an overt indication of switching between cooling and warming;
a programmable feature that allows a user to input via a graphical or digital interface any of an infinite number of therapeutic temperature and time history specifications that define a treatment protocol;
a control feature and capability to vary the therapy temperature continuously between the lowest possible value and the highest possible value; and
control hardware to implement the programmed inputs to smoothly modulate between the magnitudes of cooling and warming effects based on such standard control components as an H-bridge IC.

27.-46. (canceled)
